(19) United States
(12) Patent Application Publication
(10) Pub. No.: US 2023/0313154 A1
(43) Pub. Date: Oct. 5, 2023

(54) PRENYLTRANSFERASE ENZYMES

(71) Applicant: CB THERAPEUTICS, INC., Carlsbad, CA (US)

(72) Inventors: Erin Marie Scott, Encinitas, CA (US); Jacob Michael Vogan, San Diego, CA (US); Tyrone Jacob Yacoub, Murrieta, CA (US)

(73) Assignee: CB THERAPEUTICS, INC., Carlsbad, CA (US)

(21) Appl. No.: 18/016,673

(22) PCT Filed: Jul. 16, 2021
(86) PCT No.: PCT/US2021/042090

§ 371 Date: Jan. 17, 2023

Related U.S. Application Data
(60) Provisional application No. 63/053,539, filed on Jul. 17, 2020.

(51) Int. Cl. C12N 9/10; C12N 15/81

(52) U.S. Cl. CPC C12N 9/1085; C12N 15/81

(57) ABSTRACT

Provided is a nucleic acid comprising a sequence encoding a prenyltransferase (PT) gene or its complement, codon optimized for production in a microorganism or a plant. Also provided is a yeast expression cassette comprising the above nucleic acid. Additionally provided is a non-naturally occurring prenyltransferase (PT) comprising an amino acid sequence having at least 90% amino acid sequence identity or conservative amino acid substitutions to the amino acid sequences encoded by the above nucleic acid. Further provided is a recombinant microorganism or plant expressing a PT encoded by the above nucleic acid. Additionally provided is a method of catalyzing the condensation of a polyprenol diphosphate and an alkylresorcinol or alkylresorcyclic acid to yield a cannabinoid.

### CROSS-REFERENCE TO RELATED APPLICATIONS

This application claims the benefit of U.S. Provisional Application No. 63/053,539, filed Jul. 17, 2020, and incorporated by reference herein in its entirety.

### INCORPORATION-BY-REFERENCE OF MATERIAL SUBMITTED ON A COMPACT DISC

The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The subject matter of the Sequence Listing is incorporated herein by reference in its entirety.

### BACKGROUND OF THE INVENTION

### Field of the Invention

The present application generally relates to recombinant enzymes and genes encoding those enzymes. More specifically, the application provides recombinant prenyltransferase genes and enzymes that function in microorganisms and plants.

### Description of the Related Art

Cannabinoids are a class of organic small molecules of meroterpenoid structures found in the plant genus Cannabis. The small molecules are currently under investigation as therapeutic agents for a wide variety of health issues, including epilepsy, pain, and other neurological problems, and mental health conditions such as depression, PTSD, opioid addiction, and alcoholism (Committee on the Health Effects of Marijuana, 2017).

While it is known that cannabinoids may be obtained via biosynthesis in plant species, there are many problems associated with the synthesis of such molecules which need to be overcome, including problems with large-scale manufacturing, purification, and heterologous expression for biosynthesis.

Producing cannabinoids, in recombinant microorganisms such as yeast is a promising solution to the above problems. See, e.g., U.S. Pat. Applications 16/553103, 16/553120, 17/068636 and 63/053539; U.S. Pats. 10,435,727 and 11,041,002; and U.S. Pat. Publications 2020/0063170 and 2020/0063171, all incorporated by reference.

One way to improve biosynthetic cannabinoid production in microorganisms is by the discovery and use of new enzymes that catalyze the same reactions as plant derived enzymes but with improved parameters. The present invention provides such enzymes with prenyltransferase activity.

### BRIEF SUMMARY OF THE INVENTION

Provided is a nucleic acid comprising a sequence encoding a prenyltransferase (PT) gene or its complement, codon optimized for production in a microorganism or a plant.

Also provided is a yeast expression cassette comprising the above nucleic acid.

Additionally provided is a non-naturally occurring prenyltransferase (PT) comprising an amino acid sequence having at least 90% amino acid sequence identity or conservative amino acid substitutions to the amino acid sequences encoded by the above nucleic acid.

Further provided is a recombinant microorganism or plant expressing a PT encoded by the above nucleic acid.

The present invention is also directed to a method of catalyzing the condensation of a polyprenol diphosphate and an alkylresorcinol or alkylresorcyclic acid to yield a cannabinoid. The method comprises combining the polyprenol diphosphate and the alkylresorcinol or alkylresorcyclic acid with the above PT.

### BRIEF DESCRIPTION OF THE SEVERAL VIEWS OF THE DRAWINGS

Those of skill in the art will understand that the drawings, described below, are for illustrative purposes only. The drawings are not intended to limit the scope of the present teachings in any way.

FIG. 1 depicts a table of amino acids and codon triplets.

FIG. 2A depicts the biosynthesis pathway that yields cannabinoids and intermediates used in cannabinoid biosynthesis.

FIG. 2B depicts the mechanism for yielding cannabinoids in the heterologous recombinant host.

FIG. 3 depicts a heat cluster map of the fungal prenyltransferase enzymes and their similarity to each other.

FIG. 4 depicts a heterologous recombinant host organism expressing the fungal prenyltransferase cannabinoid pathway with and without a mixture of plant or fungal prenyltransferases.

FIG. 5 depicts the chromatogram and UV-VIS spectrum of CBGA produced by a fungal derived prenyltransferase in a recombinant host.

FIG. 6 depicts the overlay chromatogram and UV-VIS spectrum of CBGA produced by a recombinant host expressing a mixture of prenyltransferase enzymes.

FIG. 7 depicts the chromatogram and UV-VIS spectrum of THCA produced by a recombinant host expressing a fungal prenyltransferase with downstream cannabinoid synthases.

FIG. 8 depicts the chromatogram and UV-VIS spectrum of CBGVA and THCVA produced by a recombinant host expressing a fungal prenyltransferase with downstream cannabinoid synthases.

### DETAILED DESCRIPTION OF THE INVENTION

### Abbreviations and Definitions

To facilitate understanding of the invention, a number of terms and abbreviations as used herein are defined below as follows:

Conservative amino acid substitutions: As used herein, when referring to mutations in a protein, “conservative amino acid substitutions” are those in which at least one amino acid of the polypeptide encoded by the nucleic acid sequence is substituted with another amino acid having similar characteristics. Examples of conservative amino acid substitutions are ser for ala, thr, or cys; lys for arg; gln for asn, his, or lys; his for asn; glu for asp or lys; asn for his or gln; asp for glu; pro for gly; leu for ile, phe, met, or val; val for ile or leu; ile for leu, met, or val; arg for lys; met for phe; tyr for phe or trp; thr for ser; trp for tyr; and phe for tyr.

Functional variant: The term “functional variant,” as used herein, refers to a recombinant enzyme such as a fungal prenyltransferase that comprises a nucleotide and/or amino acid sequence that is altered by one or more nucleotides and/or amino acids compared to the nucleotide and/or amino acid sequences of the parent protein and that is still capable of performing an enzymatic function (e.g., synthesis of a cannabinoid) of the parent enzyme. In other words, the modifications in the amino acid and/or nucleotide sequence of the parent enzyme may cause desirable changes in reaction parameters without altering fundamental enzymatic function encoded by the nucleotide sequence or containing the amino acid sequence. The functional variant may have conservative change including nucleotide and amino acid substitutions, additions and deletions. These modifications can be introduced by standard techniques known in the art, such as site-directed mutagenesis and random PCR-mediated mutagenesis, and may comprise natural as well as non-natural nucleotides and amino acids. Also envisioned is the use of amino acid analogs, e.g. amino acids not DNA or RNA encoded in biological systems, and labels such as fluorescent dyes, radioactive elements, electron dense agents, or any other protein modification, now known or later discovered.

Recombinant nucleic acid and recombinant protein: As used herein, a recombinant nucleic acid or protein is a nucleic acid or protein produced by recombinant DNA technology, e.g., as described in Green and Sambrook (2012).

Polypeptide, protein, and peptide: The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, and the like. Modifications also include intra-molecular crosslinking and covalent attachment of various moieties such as lipids, flavin, biotin, polyethylene glycol or derivatives thereof, and the like. In addition, modifications may also include protein cyclization, branching of the amino acid chain, and cross-linking of the protein. Further, amino acids other than the conventional twenty amino acids encoded by genes may also be included in a polypeptide.

The term “protein” or “polypeptide” may also encompass a “purified” polypeptide that is substantially separated from other polypeptides in a cell or organism in which the polypeptide naturally occurs (e.g., 30%, 40%, 50%, 60%, 70%, 80%, 90%, 95%, 96%, 97%, 98%, 99%, 100% free of contaminants).

Primer, probe and oligonucleotide: The terms “primer,” “probe,” and “oligonucleotide” may be used herein interchangeably to refer to a relatively short nucleic acid fragment or sequence. They can be DNA, RNA, or a hybrid thereof, or chemically modified analogs or derivatives thereof. Typically, they are single-stranded. However, they can also be double-stranded having two complementing strands that can be separated apart by denaturation. In certain aspects, they are of a length of from about 8 nucleotides to about 200 nucleotides. In other aspects, they are from about 12 nucleotides to about 100 nucleotides. In additional aspects, they are about 18 to about 50 nucleotides. They can be labeled with detectable markers or modified in any conventional manners for various molecular biological applications.

Vector: As used herein, the term “vector” refers to a nucleic acid molecule capable of transporting another nucleic acid to which it has been linked. One type of vector is an episome, i.e., a nucleic acid capable of extra-chromosomal replication. Various vectors are those capable of autonomous replication and/expression of nucleic acids to which they are linked. Vectors capable of directing the expression of genes to which they are operatively linked are referred to herein as “expression vectors.”

Linker: The term “linker” refers to a short amino acid sequence that separates multiple domains of a polypeptide. In some embodiments, the linker prohibits energetically or structurally unfavorable interactions between the discrete domains.

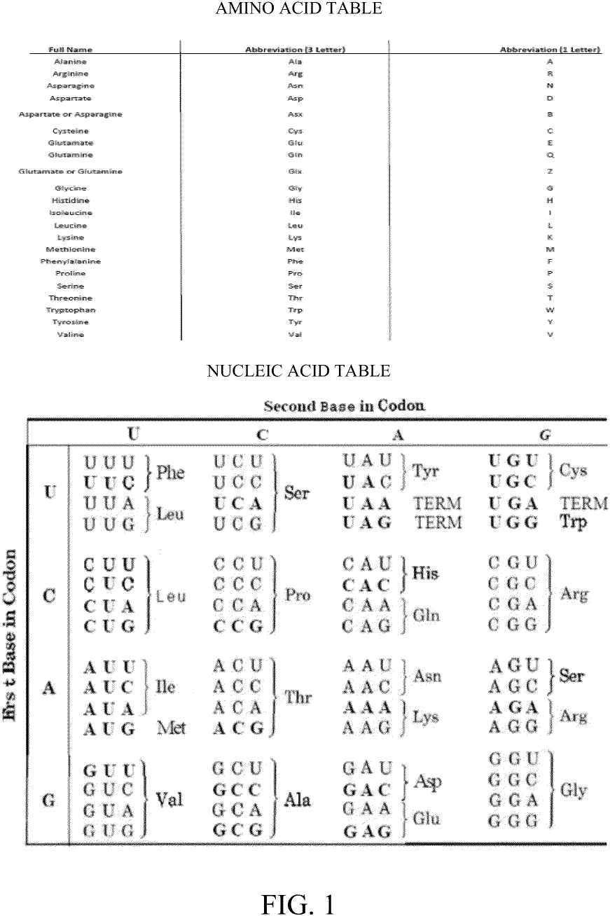

Cannabinoid: As used herein, the term “cannabinoid” refers to a family of structurally related aromatic meroterpenoid molecules. Cannabinoids are generally formed by the enzymatic fusion, by a cannabinoid synthase (having geranylpyrophosphate:olivetolate geranyltransferase activity), of an alkylresorcylic acid

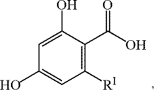

where R¹ = CH₃, (CH₂)₂CH₃ (divarinolic acid), (CH₂)₄CH₃ (olivetolic acid), or (CH₂)₆CH₃, with a polyprenyl pyrophosphate such as geranyl pyrophosphate, neryl pyrophosphate, geranylgeranyl pyrophosphate, of farnesyl pyrophosphate (FIG. 1; see also Luo et al., 2019; Carvalho et al., 2017; and Gülck and Møller, 2020 and references cited therein). The polyprenyl pyrophosphate is synthesized by geranyl pyrophosphate synthase (GPPS) (US Provisional Patent Application 63/141486).

Codon optimized: As used herein, a recombinant gene is “codon optimized” when its nucleotide sequence is modified to accommodate codon bias of the host organism to improve gene expression and increase translational efficiency of the gene.

Expression cassette: As used herein, an “expression cassette” is a nucleic acid that comprises a gene and a regulatory sequence operatively coupled to the gene such that the promoter drives the expression of the gene in a cell. An example is a gene for an enzyme with a promoter functional in yeast, where the promoter is situated such that the promoter drives the expression of the enzyme in a yeast cell.

### Prenyltransferase Enzymes for Biosynthesis of Cannabinoids

The present teachings relate to prenyltransferase (PT) enzymes, for example fungal PT enzymes, for biosynthesis of cannabinoids, nucleic acids encoding the PTs, host organisms for generating cannabinoids, and methods of generating cannabinoids in those host organisms.

PTs transfer a prenyl group, a product of the mevalonate pathway, onto a prenyl acceptor, usually a phenolic molecule, in this case, an alkylresorcylic acid, such as olivetolic acid. PTs are found in fungi, bacteria, plants, archaea, and animals and are often but not exclusively involved in secondary metabolism. PTs are also involved in basic cellular metabolism such as the biosynthesis of the cofactor Coenzyme Q.

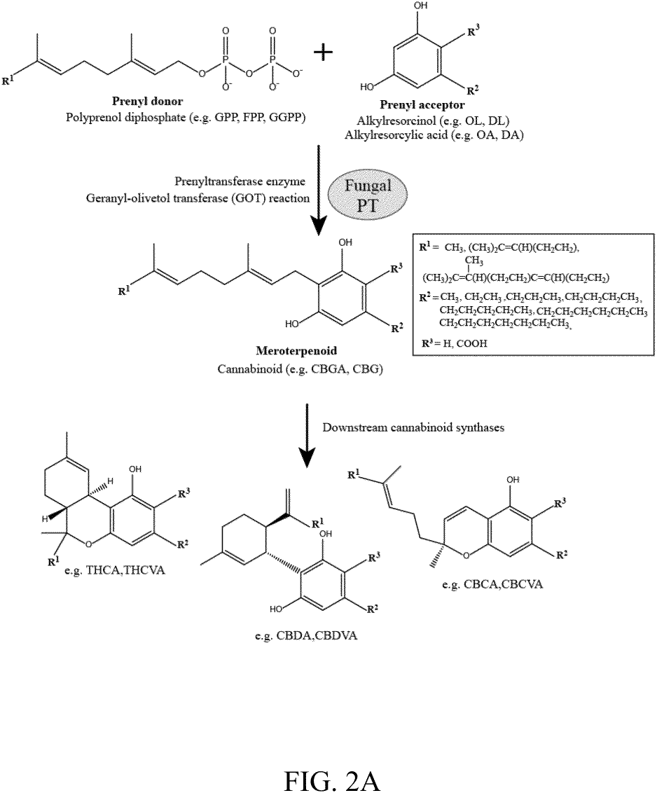

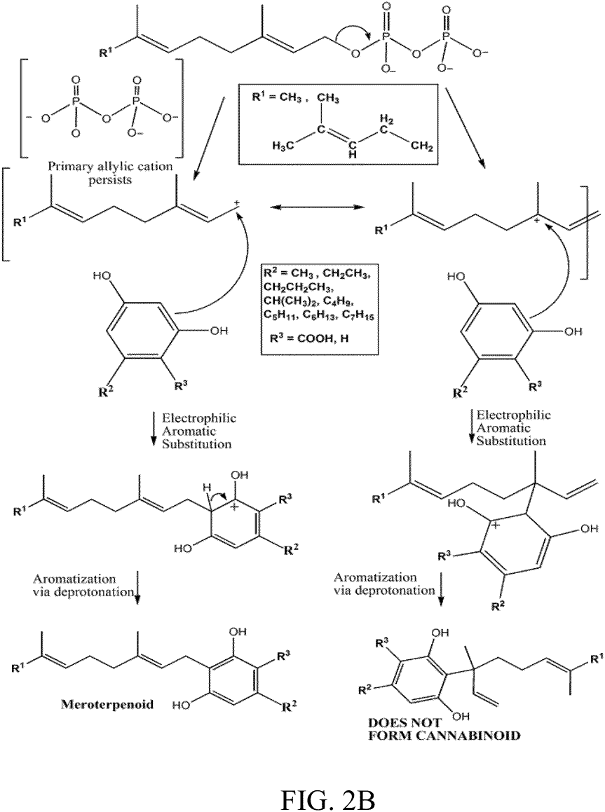

As depicted in FIG. 2A, PTs catalyze the addition of a prenyl group from a prenyl donor (e.g., GPP) containing a pyrophosphate moiety to a prenyl acceptor (e.g., OA). An ionization-condensation-elimination mechanism has been proposed as the reaction mechanism for prenyl addition. PTs may be soluble enzymes, free floating in the cytosol, or membrane integral proteins. Membrane integral proteins are distinguished by their structure, having alpha helices that pass through cellular membranes, such that the protein is embedded in either an organellar membrane or the plasma membrane of the cell.

The PTs and nucleic acids encoding the PTs are useful for producing cannabinoids genetically modified microorganisms or plants, since the PTs catalyze the condensation of olivetol (OL) or olivetolic acid (OA) and its derivatives with geranyl pyrophosphate (GPP) or other related isoprenes to yield cannabigerolic acid (CBGA), cannabigerol (CBG), cannabigerovarinic acid (CBGVA), or other cannabinoid derivatives. This engineered prenyltransferase pathway can be used in conjunction with other recombinant enzymes to make a variety of cannabinoids such as cannabidiol (CBD), tetrahydrocannabivarin (THCV), cannabichromene (CBC), and other common or novel cannabinoids.

Referring to FIG. 2A, the biosynthesis pathway, including precursors, that yields cannabinoids, is depicted. Specific cannabinoids and terpenes can be produced by varying the concentration of a precursor molecule. For example, cannabinoids produced by the metabolic pathway commencing with olivetolic acid (OA), divarinic acid (DA) (also called divarinolic acid or varinolic acid), orsellinic acid; or sphaerophorolic acid; and terpenes can be produced by the metabolic pathway commencing with geranyl pyrophosphate (GPP), farnesyl pyrophosphate (FPP), and/or geranylgeranyl pyrophosphate (GGPP).

A specific PT is the CBGA synthase/geranyl-olivetol transferase (GOT) enzyme.

FIG. 2B provides details of the PT reaction. Reverse prenylation is when the prenyl group is attached to the prenyl acceptor via a carbon-carbon bond between the prenyl acceptor and the non-pyrophosphate linked end of the prenyl donor. When pyrophosphate leaves GPP, a cation results, where the dominant species is a conical structure with the most stabilized cation (e.g., benzylic cations over secondary cations). However, other stabilized cations may be present, such as tertiary or allylic cations. This leads to a mixture of products, as there a plurality of stabilized cations, which are often reactive. The prenyl cation upon the loss of pyrophosphate may be a primary allylic cation or a tertiary allylic cation (via the migration of the terminal double bond for resonance stabilization). Tertiary allylic cations are able to stabilize the positive charge with both inductive and resonance stabilizations. Primary allylic cations are able to stabilize the positive charge with resonance stabilization. Therefore, the tertiary allylic cation is more stabilized than the primary allylic cation. As such, the tertiary allylic cation is expected to persist in higher concentrations than the primary cation. However, there is no evidence of the tertiary allylic cation persisting or even forming as the reaction pathway on the right side of FIG. 2B does not occur in the formation of classic cannabinoids (e.g. CBGA) and meroterpenoids. In contrast, the primary allylic cation on the left side of FIG. 2B exclusively persists for electrophilic aromatic substitution and subsequent deprotonative aromatization to selectively form meroterpenoids, as catalyzed by fungal PT in the recombinant host.

Non-Cannabis derived PTs can: (i) catalyze reverse prenylations, or (ii) have O-specificity or N-specificity. Some PTs are specific for prenylating proteins, attaching the prenyl group to a cysteine residue in the C-terminus of the target protein. O-specificity is where the prenyl group is attached to an oxygen in the prenyl acceptor instead of a carbon. N-specificity is when the prenyl group is attached to a nitrogen in the prenyl acceptor instead of a carbon. The nitrogen of the indole ring on a tryptophan residue is another possible target for protein prenylation. Some PTs are specific in their requirements for the prenyl donor.

Cannabis-derived PTs may use: (i) GPP (10 carbon prenyl donor); (ii) DMAPP (5 carbon prenyl donor); (iii) FPP (15 carbon prenyl donor); or (iv) GGPP (20 carbon prenyl donor). Only a limited subset of Cannabis-derived PTs are able to produce cannabinoids.

It should be noted that not all PTs can synthesize cannabinoids. Many PTs have loose substrate specificity and can accept a wide variety of substrates, but cannabinoid biosynthesis activity is rare in nature. Cannabis spp. are the primary organisms known to produce cannabinoids in nature.

Precursors of cannabinoids include alkylrescorcinol and alkylrescorcyclic acid compounds such as olivetol (OL) and olivetolic acid (OA) that are produced from common cellular metabolites hexanoyl-CoA and malonyl CoA.

In plants, olivetol is produced from hexanoyl-CoA and malonyl-CoA by the action of the polyketide synthase olivetol synthase; and olivetolic acid is produced from olivetol by the action of the enzyme olivetolic acid cyclase. See, e.g., US Provisional Patent Application 63/194,121.

Another precursor compound is a polyprenol compound, geranyl pyrophosphate (GPP) (also referred to as geranyl diphosphate), which is a product of the mevalonate pathway (see FIG. 2A). See, e.g., U.S. Provisional Pat. Application 63/141,486.

In plants, cannabigerolic acid (CBGA) is produced from OA and GPP by the action of the enzyme cannabigerolic acid synthase (CBGAS), also called geranyl-olivetol transferase (GOT).

Downstream cannabinoids are produced by the action of downstream cannabinoid synthases on cannabigerolic acid. For example, THCA synthase converts CBGA to THCA. Cannabidiolic acid synthase converts CBGA to CBDA. Cannabichromenic acid synthase converts CBGA to CBCA (see FIG. 2A). Other cannabinoids are made by further modification(s) of CBGA, CBGVA, CBGOA, CBGPA, CBDA, THCA, and/or CBCA.

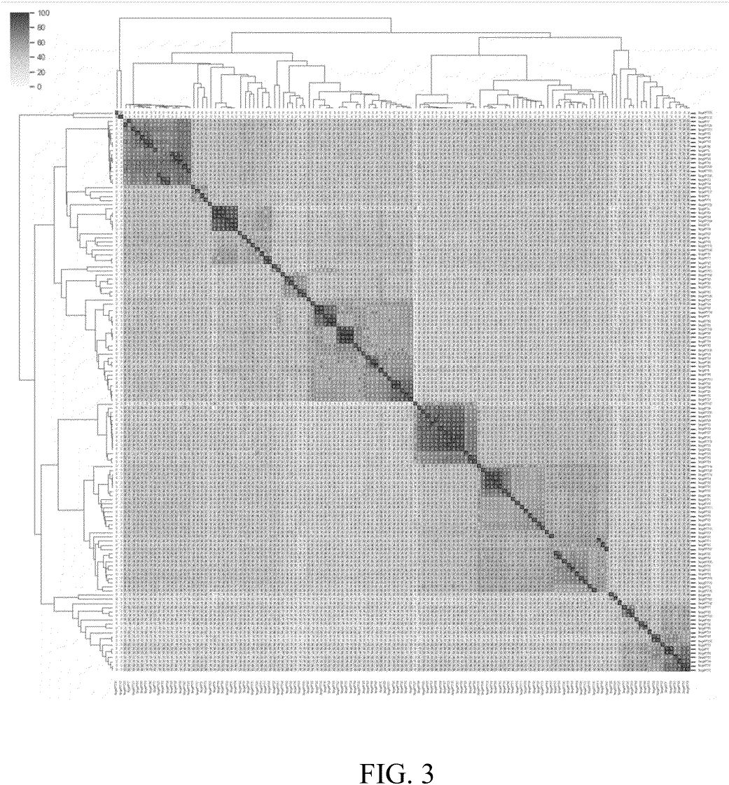

Referring to FIG. 3, a two-dimensional clustermap demonstrates the diversity of fungal PT enzymes with GOT activity, which displays the percentage of identical residues between each pair of Fungal PT enzymes, where a darker shaded block correlates with higher similarity.

The activity of the putative PTs can be determined by any method known in the art. In some embodiments, each candidate polypeptide is introduced into a host cell genetically modified to contain the components for CBGA biosynthesis using standard yeast cell transformation techniques (see, e.g., Green and Sambrook, 2012). Cells are subjected to fermentation, under conditions that activate the promoter controlling the candidate polypeptide (see Example 2). The fermentation broth is then subjected to HPLC analysis (see Example 5). As an example of GOT function determination of the resulting polypeptide, if CBGA is detected by HPLC analysis relative to an analytical standard, then the candidate polypeptide is determined to be a functional CBGA synthase in this context.

### Nucleic Acids

In some embodiments, a nucleic acid comprising a sequence encoding a prenyltransferase (PT) gene or its complement is provided. In these embodiments, the PT gene is codon optimized for production in a microorganism or a plant.

As provided in US Pat. No. 10,435,727, nucleotide sequences are codon optimized for expression of heterologous polypeptides in a host by utilizing a number of considerations:
(1) For each amino acid of the recombinant polypeptide to be expressed, a codon (triplet of nucleotide bases) is selected based on the frequency of each codon in a heterologous host microorganism or plant genome, for example the Saccharomyces cerevisiae genome; the codon can be chosen to be the most frequent codon or can be selected probabilistically based on the frequencies of all possible codons.
(2) In order to prevent DNA cleavage due to a restriction enzyme, certain restriction sites are removed by changing codons that cover those sites.
(3) To prevent low-complexity regions, long repeats (sequences of any single base longer than five bases) are modified. (2) and (3) are performed recursively to ensure that codon modification does not lead to additional undesirable sequences.
(4) A ribosome binding site is added to the N-terminus.
(5) A stop codon is added.
(6) Codons coding for amino acids prone to undesirable post-translational modifications are mutated, such as changing protein surface exposed lysine codons to arginine codons to prevent protein ubiquitination.

Enzyme variations may also result from mutations introduced into the DNA and amino acid sequences to prevent or promote post translational modifications of the protein. Nonlimiting examples of post translational modifications include phosphorylation, acetylation, methylation, SUMOylation, ubiquitination, proteolytic cleavage, lipidation, including prenylation such as farnesylation or myristoylation, glycosylation, nitrosylation and biotinylation.

In various embodiments, the gene for the PT enzyme is derived from a fungus. It is envisioned that a PT from any fungus now known or later discovered can be utilized in the present invention. This includes but is not limited to the phyla Chytridiomycota, Basidiomycota, Ascomycota, Blastocladiomycota, Ascomycota, Microsporidia, Basidiomycota, Glomeromycota, Symbiomycota, and Neocallimastigomycota. For example, the fungus can be from the phylum Ascomycota, including classes, orders, families and genera Pezizomycotina, Arthoniomycetes, Colletotrichum, Coniocybomycetes, Clavicipitaceae, Dothideomycetes, Eurotiomycetes, Geoglossomycetes, Glomerellales, Hypocreales Laboulbeniomycetes, Lecanoromycetes, Leotiomycetes, Lichinomycetes, Orbiliomycetes, Pezizomycetes, Sordariomycetes, Xylonomycetes, Lahmiales, Itchiclahmadion, Triblidiales, Saccharomycotina, Saccharomycetes, Stachybotryaceae, Taphrinomycotina, Archaeorhizomyces, Neolectomycetes, Pneumocystidomycetes, Schizosaccharomycetes, and Taphrinomycetes; phylum Basidiomycota including subphyla or classes Pucciniomycotina, Ustilaginomycotina, Wallemiomycetes, and Entorrhizomycetes; subphylum Agaricomycotina including classes Tremellomycetes, Dacrymycetes, and Agaricomycetes; phylum Symbiomycota, including class Entorrhizomycota; subphylum Ustilaginomycotina including classes Ustilaginomycetes and Exobasidiomycetes; phylum Glomeromycota including classes Archaeosporomycetes, Glomeromycetes, and Paraglomeromycetes; subphylum Pucciniomycotina including orders and classes: Pucciniomycotina, Cystobasidiomycetes, Agaricostilbomycetes, Microbotryomycetes, Atractiellomycetes, Classiculomycetes, Mixiomycetes, and Cryptomycocolacomycetes; subphylum incertae sedis Mucoromyceta including orders Calcarisporiellomycota and Mucoromycota; phylum Mortierellomyceta including class Mortierellomycota; subphylum incertae sedis Entomophthoromycotina including order Entomophthorales; phylum Zoopagomyceta including classes Basidiobolomycota, Entomophthoromycota, Kickxellomycota, and Zoopagomycotina; subphylum incertae sedis Mucoromycotina including orders Mucorales, Endogonales, and Mortierellales; phylum Neocallimastigomycota including class Neocallimastigomycetes; phylum Blastocladiomycota including classes Physodermatomycetes and Blastocladiomycetes; phylum Rozellomyceta including classes Rozellomycota and Microsporidia; phylum Aphelidiomyceta including class Aphelidiomycota; Chytridiomyceta including classes Chytridiomycetes and Monoblepharidomycetes; and phylum Oomycota including classes or orders Leptomitales, Myzocytiopsidales, Olpidiopsidales, Peronosporales, Pythiales, Rhipidiales, Salilagenidiales, Saprolegniales, Sclerosporales, Anisolpidiales, Lagenismatales, Rozellopsidales, and Haptoglossales.

Specific modified fungal PT enzymes presented herein have the capacity to synthesize cannabinoids such as CBGA in a recombinant host. SEQ ID NOs: 1-135 are codon optimized nucleic acid sequences encoding modified fungal PTs having amino acid sequences SEQ ID NOs: 136-270 (Table 1).

TABLE 1

Fungal PT Genes for Nucleic Acids Corresponding to the Encoded Isolated Proteins

| | | |
|---|---|---|
| Shorthand | Codon Optimized Nucleic Acid Sequence | Amino Acid Sequence for Isolated Protein |
| FungalPT1 | SEQ. ID NO: 1 | SEQ. ID NO: 136 |
| FungalPT2 | SEQ. ID NO: 2 | SEQ. ID NO: 137 |
| FungalPT3 | SEQ. ID NO: 3 | SEQ. ID NO: 138 |
| FungalPT4 | SEQ. ID NO: 4 | SEQ. ID NO: 139 |
| FungalPT5 | SEQ. ID NO: 5 | SEQ. ID NO: 140 |
| FungalPT6 | SEQ. ID NO: 6 | SEQ. ID NO: 141 |
| FungalPT7 | SEQ. ID NO: 7 | SEQ. ID NO: 142 |
| FungalPT8 | SEQ. ID NO: 8 | SEQ. ID NO: 143 |
| FungalPT9 | SEQ. ID NO: 9 | SEQ. ID NO: 144 |
| FungalPT10 | SEQ. ID NO: 10 | SEQ. ID NO: 145 |
| FungalPT11 | SEQ. ID NO: 11 | SEQ. ID NO: 146 |
| FungalPT12 | SEQ. ID NO: 12 | SEQ. ID NO: 147 |
| FungalPT13 | SEQ. ID NO: 13 | SEQ. ID NO: 148 |
| FungalPT14 | SEQ. ID NO: 14 | SEQ. ID NO: 149 |
| FungalPT15 | SEQ. ID NO: 15 | SEQ. ID NO: 150 |
| FungalPT16 | SEQ. ID NO: 16 | SEQ. ID NO: 151 |
| FungalPT17 | SEQ. ID NO: 17 | SEQ. ID NO: 152 |
| FungalPT18 | SEQ. ID NO: 18 | SEQ. ID NO: 153 |
| FungalPT19 | SEQ. ID NO: 19 | SEQ. ID NO: 154 |
| FungalPT20 | SEQ. ID NO: 20 | SEQ. ID NO: 155 |
| FungalPT21 | SEQ. ID NO: 21 | SEQ. ID NO: 156 |
| FungalPT22 | SEQ. ID NO: 22 | SEQ. ID NO: 157 |
| FungalPT23 | SEQ. ID NO: 23 | SEQ. ID NO: 158 |
| FungalPT24 | SEQ. ID NO: 24 | SEQ. ID NO: 159 |
| FungalPT25 | SEQ. ID NO: 25 | SEQ. ID NO: 160 |
| FungalPT26 | SEQ. ID NO: 26 | SEQ. ID NO: 161 |
| FungalPT27 | SEQ. ID NO: 27 | SEQ. ID NO: 162 |
| FungalPT28 | SEQ. ID NO: 28 | SEQ. ID NO: 163 |
| FungalPT29 | SEQ. ID NO: 29 | SEQ. ID NO: 164 |
| FungalPT30 | SEQ. ID NO: 30 | SEQ. ID NO: 165 |
| FungalPT31 | SEQ. ID NO: 31 | SEQ. ID NO: 166 |
| FungalPT32 | SEQ. ID NO: 32 | SEQ. ID NO: 167 |
| FungalPT33 | SEQ. ID NO: 33 | SEQ. ID NO: 168 |
| FungalPT34 | SEQ. ID NO: 34 | SEQ. ID NO: 169 |
| FungalPT35 | SEQ. ID NO: 35 | SEQ. ID NO: 170 |
| FungalPT36 | SEQ. ID NO: 36 | SEQ. ID NO: 171 |
| FungalPT37 | SEQ. ID NO: 37 | SEQ. ID NO: 172 |
| FungalPT38 | SEQ. ID NO: 38 | SEQ. ID NO: 173 |
| FungalPT39 | SEQ. ID NO: 39 | SEQ. ID NO: 174 |
| FungalPT40 | SEQ. ID NO: 40 | SEQ. ID NO: 175 |
| FungalPT41 | SEQ. ID NO: 41 | SEQ. ID NO: 176 |
| FungalPT42 | SEQ. ID NO: 42 | SEQ. ID NO: 177 |
| FungalPT43 | SEQ. ID NO: 43 | SEQ. ID NO: 178 |
| FungalPT44 | SEQ. ID NO: 44 | SEQ. ID NO: 179 |
| FungalPT45 | SEQ. ID NO: 45 | SEQ. ID NO: 180 |
| FungalPT46 | SEQ. ID NO: 46 | SEQ. ID NO: 181 |
| FungalPT47 | SEQ. ID NO: 47 | SEQ. ID NO: 182 |
| FungalPT48 | SEQ. ID NO: 48 | SEQ. ID NO: 183 |
| FungalPT49 | SEQ. ID NO: 49 | SEQ. ID NO: 184 |
| FungalPT50 | SEQ. ID NO: 50 | SEQ. ID NO: 185 |
| FungalPT51 | SEQ. ID NO: 51 | SEQ. ID NO: 186 |
| FungalPT52 | SEQ. ID NO: 52 | SEQ. ID NO: 187 |
| FungalPT53 | SEQ. ID NO: 53 | SEQ. ID NO: 188 |
| FungalPT54 | SEQ. ID NO: 54 | SEQ. ID NO: 189 |
| FungalPT55 | SEQ. ID NO: 55 | SEQ. ID NO: 190 |
| FungalPT56 | SEQ. ID NO: 56 | SEQ. ID NO: 191 |
| FungalPT57 | SEQ. ID NO: 57 | SEQ. ID NO: 192 |
| FungalPT58 | SEQ. ID NO: 58 | SEQ. ID NO: 193 |
| FungalPT59 | SEQ. ID NO: 59 | SEQ. ID NO: 194 |
| FungalPT60 | SEQ. ID NO: 60 | SEQ. ID NO: 195 |
| FungalPT61 | SEQ. ID NO: 61 | SEQ. ID NO: 196 |
| FungalPT62 | SEQ. ID NO: 62 | SEQ. ID NO: 197 |
| FungalPT63 | SEQ. ID NO: 63 | SEQ. ID NO: 198 |
| FungalPT64 | SEQ. ID NO: 64 | SEQ. ID NO: 199 |
| FungalPT65 | SEQ. ID NO: 65 | SEQ. ID NO: 200 |
| FungalPT66 | SEQ. ID NO: 66 | SEQ. ID NO: 201 |
| FungalPT67 | SEQ. ID NO: 67 | SEQ. ID NO: 202 |
| FungalPT68 | SEQ. ID NO: 68 | SEQ. ID NO: 203 |
| FungalPT69 | SEQ. ID NO: 69 | SEQ. ID NO: 204 |
| FungalPT70 | SEQ. ID NO: 70 | SEQ. ID NO: 205 |
| FungalPT71 | SEQ. ID NO: 71 | SEQ. ID NO: 206 |
| FungalPT72 | SEQ. ID NO: 72 | SEQ. ID NO: 207 |
| FungalPT73 | SEQ. ID NO: 73 | SEQ. ID NO: 208 |
| FungalPT74 | SEQ. ID NO: 74 | SEQ. ID NO: 209 |
| FungalPT75 | SEQ. ID NO: 75 | SEQ. ID NO: 210 |
| FungalPT76 | SEQ. ID NO: 76 | SEQ. ID NO: 211 |
| FungalPT77 | SEQ. ID NO: 77 | SEQ. ID NO: 212 |
| FungalPT78 | SEQ. ID NO: 78 | SEQ. ID NO: 213 |
| FungalPT79 | SEQ. ID NO: 79 | SEQ. ID NO: 214 |
| FungalPT80 | SEQ. ID NO: 80 | SEQ. ID NO: 215 |
| FungalPT81 | SEQ. ID NO: 81 | SEQ. ID NO: 216 |
| FungalPT82 | SEQ. ID NO: 82 | SEQ. ID NO: 217 |
| FungalPT83 | SEQ. ID NO: 83 | SEQ. ID NO: 218 |
| FungalPT84 | SEQ. ID NO: 84 | SEQ. ID NO: 219 |
| FungalPT85 | SEQ. ID NO: 85 | SEQ. ID NO: 220 |
| FungalPT86 | SEQ. ID NO: 86 | SEQ. ID NO: 221 |
| FungalPT87 | SEQ. ID NO: 87 | SEQ. ID NO: 222 |
| FungalPT88 | SEQ. ID NO: 88 | SEQ. ID NO: 223 |
| FungalPT89 | SEQ. ID NO: 89 | SEQ. ID NO: 224 |
| FungalPT90 | SEQ. ID NO: 90 | SEQ. ID NO: 225 |
| FungalPT91 | SEQ. ID NO: 91 | SEQ. ID NO: 226 |
| FungalPT92 | SEQ. ID NO: 92 | SEQ. ID NO: 227 |
| FungalPT93 | SEQ. ID NO: 93 | SEQ. ID NO: 228 |
| FungalPT94 | SEQ. ID NO: 94 | SEQ. ID NO: 229 |
| FungalPT95 | SEQ. ID NO: 95 | SEQ. ID NO: 230 |
| FungalPT96 | SEQ. ID NO: 96 | SEQ. ID NO: 231 |
| FungalPT97 | SEQ. ID NO: 97 | SEQ. ID NO: 232 |
| FungalPT98 | SEQ. ID NO: 98 | SEQ. ID NO: 233 |
| FungalPT99 | SEQ. ID NO: 99 | SEQ. ID NO: 234 |
| FungalPT100 | SEQ. ID NO: 100 | SEQ. ID NO: 235 |
| FungalPT101 | SEQ. ID NO: 101 | SEQ. ID NO: 236 |
| FungalPT102 | SEQ. ID NO: 102 | SEQ. ID NO: 237 |
| FungalPT103 | SEQ. ID NO: 103 | SEQ. ID NO: 238 |
| FungalPT104 | SEQ. ID NO: 104 | SEQ. ID NO: 239 |
| FungalPT105 | SEQ. ID NO: 105 | SEQ. ID NO: 240 |
| FungalPT106 | SEQ. ID NO: 106 | SEQ. ID NO: 241 |
| FungalPT107 | SEQ. ID NO: 107 | SEQ. ID NO: 242 |
| FungalPT108 | SEQ. ID NO: 108 | SEQ. ID NO: 243 |
| FungalPT109 | SEQ. ID NO: 109 | SEQ. ID NO: 244 |
| FungalPT110 | SEQ. ID NO: 110 | SEQ. ID NO: 245 |
| FungalPT111 | SEQ. ID NO: 111 | SEQ. ID NO: 246 |
| FungalPT112 | SEQ. ID NO: 112 | SEQ. ID NO: 247 |
| FungalPT113 | SEQ. ID NO: 113 | SEQ. ID NO: 248 |
| FungalPT114 | SEQ. ID NO: 114 | SEQ. ID NO: 249 |
| FungalPT115 | SEQ. ID NO: 115 | SEQ. ID NO: 250 |
| FungalPT116 | SEQ. ID NO: 116 | SEQ. ID NO: 251 |
| FungalPT117 | SEQ. ID NO: 117 | SEQ. ID NO: 252 |
| FungalPT118 | SEQ. ID NO: 118 | SEQ. ID NO: 253 |
| FungalPT119 | SEQ. ID NO: 119 | SEQ. ID NO: 254 |
| FungalPT120 | SEQ. ID NO: 120 | SEQ. ID NO: 255 |
| FungalPT121 | SEQ. ID NO: 121 | SEQ. ID NO: 256 |
| FungalPT122 | SEQ. ID NO: 122 | SEQ. ID NO: 257 |
| FungalPT123 | SEQ. ID NO: 123 | SEQ. ID NO: 258 |
| FungalPT124 | SEQ. ID NO: 124 | SEQ. ID NO: 259 |
| FungalPT125 | SEQ. ID NO: 125 | SEQ. ID NO: 260 |
| FungalPT126 | SEQ. ID NO: 126 | SEQ. ID NO: 261 |
| FungalPT127 | SEQ. ID NO: 127 | SEQ. ID NO: 262 |
| FungalPT128 | SEQ. ID NO: 128 | SEQ. ID NO: 263 |
| FungalPT129 | SEQ. ID NO: 129 | SEQ. ID NO: 264 |
| FungalPT130 | SEQ. ID NO: 130 | SEQ. ID NO: 265 |
| FungalPT131 | SEQ. ID NO: 131 | SEQ. ID NO: 266 |
| FungalPT132 | SEQ. ID NO: 132 | SEQ. ID NO: 267 |
| FungalPT133 | SEQ. ID NO: 133 | SEQ. ID NO: 268 |
| FungalPT134 | SEQ. ID NO: 134 | SEQ. ID NO: 269 |
| FungalPT135 | SEQ. ID NO: 135 | SEQ. ID NO: 270 |

Homologous polynucleotide sequences, or complements thereof, having at least about 80%, 81%, 82%, 83%, 84%, 85%, 86%, 87%, 88%, 89%, 90%, 91%, 92%, 93%, 94%, 95%, 96%, 97%, 98%, 99% or 100% identity to the polynucleotide sequences listed sequences SEQ. ID NOs— 1-135 are also provided, including those that encode corresponding amino acid sequences SEQ ID NOs: 136-270, respectively, or having conservative amino acid substitutions. In some embodiments, the encoded PT comprises one or more conservative amino acid substitution that prevents or promotes post-translational modification of the PT. Examples of such post-translational modification is phosphorylation, acetylation, methylation, SUMOylation, ubiquitination, proteolytic cleavage, lipidation, prenylation such as farnesylation or myristoylation, glycosylation, nitrosylation, biotinylation, or any combination thereof.

In various embodiments, the nucleic acids further comprise additional nucleic acids encoding amino acids that are not part of the PT enzyme. In some of these embodiments, the additional sequences encode additional amino acids present when the nucleic acid is translated, encoding, for example, an additional protein domain, with or without a linker sequence, creating a fusion protein. Other examples are localization sequences, i.e., signals directing the localization of the folded protein to a specific subcellular compartment or membrane.

In some embodiments, the nucleic acids have, at the 5′ end, a nucleic acid encoding codon optimized cofolding peptides to create a fusion protein, e.g., having SEQ ID NOs:271-275 (Table 2), joining the sequences together to form a fusion polypeptide, e.g., having the amino acid sequence of SEQ ID NO:244-248 fused at the N terminus of the enzyme polypeptide, generating recombinant fusion polypeptides.

TABLE 2

| | | |
|---|---|---|
| NAME | Codon Optimized Nucleic Acid Sequence | Amino Acid Sequence for Isolated Protein |
| MBP | Seq. ID NO:271 | Seq. ID NO:276 |
| VEN | Seq. ID NO:272 | Seq. ID NO:277 |
| MST | Seq. ID NO:273 | Seq. ID NO:278 |
| OSP | Seq. ID NO:274 | Seq. ID NO:279 |
| OLE | Seq. ID NO:275 | Seq. ID NO:280 |

In some embodiments, the nucleic acid comprises additional nucleotide sequences that are not translated. Nonlimiting examples include promoters, terminators, barcodes, Kozak sequences, targeting sequences, and enhancer elements.

Expression of a gene encoding an enzyme is determined by the promoter controlling the gene. In order for a gene to be expressed, a promoter must be present within 1,000 nucleotides upstream of the gene. A gene is generally cloned under the control of a desired promoter. The promoter regulates the amount of enzyme expressed in the cell and also the timing of expression, or expression in response to external factors such as sugar source. Any promoter now known or later discovered can be utilized to drive the expression of the PT described herein.

In various embodiments, the nucleic acid encoding the PT is codon optimized for expression in yeast, for example a species of Saccharomyces, Candida, Pichia, Schizosaccharomyces, Scheffersomyces, Blakeslea, Rhodotorula, or Yarrowia. Such nucleic acids can usefully comprise a promoter functional in yeast. See e.g. http://parts.igem.org/Yeast for a listing of various yeast promoters. Exemplary yeast promoters known in the art are listed in Table 3 below drive strong expression, constant gene expression, medium or weak gene expression, or inducible gene expression. Inducible or repressible gene expression is dependent on the presence or absence of a certain molecule. For example, the GAL1, GAL7, and GAL10 promoters are activated by the presence of the sugar galactose and repressed by the presence of the sugar glucose. The HO promoter is active and drives gene expression only in the presence of the alpha factor peptide. The HXT1 promoter is activated by the presence of glucose while the ADH2 promoter is repressed by the presence of glucose.

TABLE 3

Exemplary yeast promoters

| | | |
|---|---|---|
| Strong constitutive promoters | Medium and weak constitutive promoters | Inducible/repressible promoters |
| TEF1 | STE2 | GAL1 |
| PGK1 | TPI1 | GAL7 |
| PGI1 | PYK1 | GAL10 |
| TDH3 | | HO |
| | | HXT1 |
| | | ADH2 |

In various embodiments, the nucleic acid is in an expression cassette, e.g., a yeast expression cassette. Any yeast expression cassette capable of expressing the enzyme in a yeast cell can be utilized. In some embodiments, the expression cassette consists of a nucleic acid encoding a PT with a promoter.

Additional regulatory elements can also be present in the expression cassette, including restriction enzyme cleavage sites, antibiotic resistance genes, integration sites, auxotrophic selection markers, origins of replication, and degrons.

The expression cassette can be present in a vector that, when transformed into a host cell, either integrates into chromosomal DNA or remains episomal in the host cell. Such vectors are well-known in the art. See e.g. http://parts.igem.org/Yeast for a listing of various yeast vectors.

A nonlimiting example of a yeast vector is a yeast episomal plasmid (YEp) that contains the pBluescript II SK(+) phagemid backbone, an auxotrophic selectable marker, yeast and bacterial origins of replication and multiple cloning sites enabling gene cloning under a suitable promoter. Other exemplary vectors include pRS series plasmids.

### Prenyltransferase Proteins

Additionally provided is a non-naturally occurring prenyltransferase (PT) having GOT PT activity. In various embodiments, the GOT PT comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity or conservative amino acid substitutions to SEQ ID NOs: 136-270.

In some embodiments, the GOT PT is provided in vitro. In other embodiments, the GOT PT is expressed in a recombinant host cell, e.g., a microorganism or a plant such as tobacco or Arabidopsis thaliana. Nonlimiting examples of host cell microorganisms include any species of bacteria, including but not limited to Escherichia, Corynebacterium, Caulobacter, Pseudomonas, Streptomyces, Bacillus or Lactobacillus; any species of filamentous fungus, including but not limited to any species of Aspergillus, and a yeast, e.g., any species of Saccharomyces, Candida, Pichia, Schizosaccharomyces, Scheffersomyces, Blakeslea, Rhodotorula or Yarrowia

### Recombinant Host Cells

Further provided is a recombinant host cell of a microorganism or plant expressing a GOT PT, e.g., encoded by any of the above-described nucleic acids.

Referring to FIG. 2A, the recombinant PT may be added to the host cell by any technique known in the art, e.g., as described in Green and Sambrook, 2012. The host organism may also express enzymes designed to increase the amount of a precursor molecule, e.g., GPP. Those enzymes may be derived from any combination of the mevalonate (MEV) pathway or the MEP pathway (methyl erythritol phosphate) pathway, e.g., as described in U.S. Pat. Application 63/194121, U.S. Pat. Application 63/164126, U.S. Pat. Application 63/141486, and U.S. Pat. 11,041,002. Non-limiting examples of such enzymes are an olivetolic acid cyclase (OAC), a polyketide synthase, an olivetol synthase, a geranyl pyrophosphate synthase (GPPS), a malonate transporter, an aromatase, a dehydrogenase, an oxidase, a desaturase, a decarboxylase, a cannabinoid synthase including a THCA, a CBCA or a CBDA synthase, a cytochrome P450 (CYP-450), a cytochrome P450 reductase (CPR), or any combination thereof.

Examples of cannabinoids that may be produced in these host cells include cannabigerol (CBG), cannabigerolic acid (CBGA), cannabidiolic acid (CBDA), cannabichromene (CBC), cannabidivarin (CBCV), cannabichromenic acid (CBCA), cannabichromevarinic acid (CBCVA), cannabinol (CBN), 11-hydroxycannabinol (11-OH-CBN), cannabinerolic acid (CBNA), cannabivarin (CBV), 11-hydroxycannabivarin (11-OH-CBV), cannabinerovarinic acid (CBNVA), cannabigerophorolic acid (CBGPA), cannabigerovarinic acid (CBGVA), cannabigerogerovarinic acid (CBGGVA), tetrahydrocannabinol (THC), tetrahydrocannabinolic acid (THCA), 11-hydroxy tetrahydrocannabinol (11-OH-THC), tetrahydrocannabivarin (THCV), 11-hydroxy tetrahydrocannabivarin (11-OH-THCV), tetrahydrocannabivarin acid (THCVA), cannabinerovarinic acid (CBNVA), sesquicannabigerol (CBF), cannabigerogerol (CBGG), sesquicannabigerolic acid (CBFA), cannabigerogerolic acid (CBGGA), sesquicannabidiolic acid (CBDFA), sesquitetrahydrocannabinolic acid (THCFA), sesquicannabigerovarinic acid (CBFVA), sesquicannabichromenic acid (CBCFA), sesquicannabigerophorolic acid (CBFPA), cannabidiol (CBD), cannabidiolic acid (CBDA), cannabidivarinic acid (CBDVA), cannabidivarin (CBDV) or any combination thereof.

Cannabinoids can be produced in a heterologous cell along the pathway depicted in FIG. 2A. The methods for improving production of precursor molecules to the cannabinoids are realized by genetically modifying the host organism.

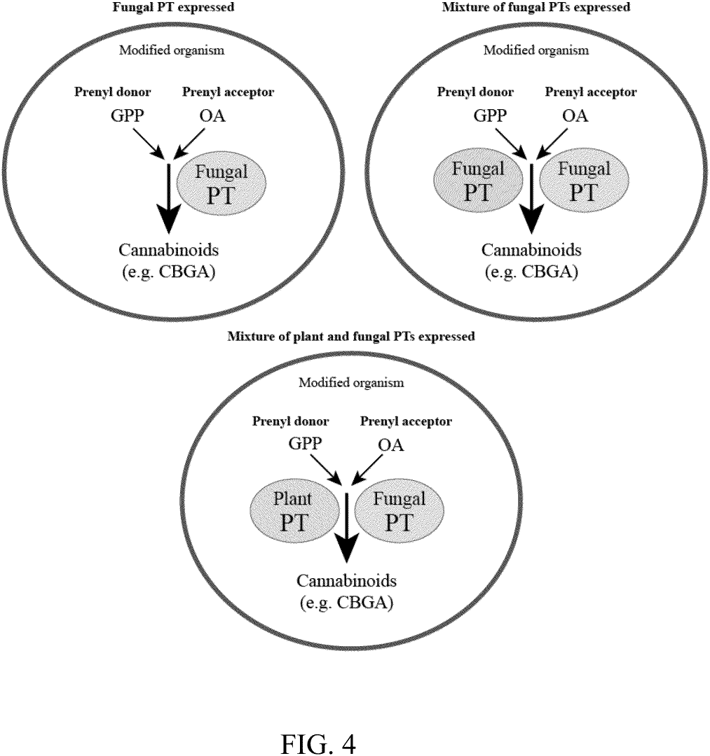

Referring to FIG. 4, DNA sequences are synthesized and cloned using techniques known in the art. Gene expression can be controlled by inducible or constitutive promoter systems using the appropriate expression vectors. Genes are transformed into an organism using standard transformation methods to generate modified host strains (i.e., the recombinant host organism). The modified strains which produce cannabinoid precursors express genes for one or more prenyltransferases, e.g., any of the GOT PTs described above, or a mixture of GOT PTs, e.g., from fungal or plant origin. Thus, one of skill in the art would recognize plant PT genes and enzymes can be linked in combination with the fungal PT genes and enzymes, such that the host cells can be transformed. The production of high value molecules in the host cells can also be determined by the methods herein. The modified strains from above can also co-express genes for downstream cannabinoids synthases, such as CBCA, THCA, and CBDA synthases, to produce additional cannabinoids compounds including but not limited to CBCA, CBCVA, CBC, THCA, THCVA, THCV, CBDA, CBDVA, CBD, etc., e.g., as described in US Provisional Patent Application 63/164126.

Thus, the host cell for producing cannabinoids can comprise one or more PT genes; a prenyl group acceptor; a prenyl group donor; and a reaction pathway for converting the prenyl group donor and the prenyl group acceptor to a first cannabinoid catalyzed by PT enzymes encoded by the PT genes.

The one or more PT genes may be inserted into and thereby integrated into the host cell and one or more PT genes are expressed. In some embodiments, the PT enzymes are at least 80% homologous to isolated amino acid sequences selected from SEQ. ID NOs: 136-270 which may be encoded by the PT genes having SEQ ID NOs:1-135, respectively.

In some embodiments, genetic alteration is achieved by: gene expression controlled by inducible promoter systems; natural or induced mutagenesis, recombination, and/or shuffling of genes, pathways, and whole cells performed sequentially or in cycles; overexpression and/or deletion of single or multiple genes; knockout mutations/alterations which reduce or eliminate parasitic pathways that reduce precursor concentration; and integrating vectors and PCR fragments into the host genome.

In other embodiments, the recombinant host cells of the recombinant organism are engineered to produce all precursor molecules necessary for the biosynthesis of cannabinoids, including but not limited to OA, OL and GPP, hexanoic acid, hexanoyl-CoA, malonic acid and malonyl-CoA, e.g., as disclosed in U.S. Pat. No. 10,435,727.

As discussed above, in some embodiments, the recombinant host cell is a yeast cell, e.g., a species of Saccharomyces, Candida, Pichia, Schizosaccharomyces, Scheffersomyces, Blakeslea, Rhodotorula, or Yarrowia.

Using the systems and methods herein, the genes which can be expressed to encode for a corresponding enzyme or other type of proteins include but are not limited to the fungal prenyltransferases (Fungal PT). For example, the Fungal PT gene is expressed, or overexpressed, to encode for the Fungal PT enzyme. Gene sequences can be determined using the techniques disclosed in U.S. Pat. 10,671,632.

In various embodiments, samples from fermentations of recombinant hosts expressing the cannabinoid pathway with the GOT PTs described above are: (i) prepared and extracted using a combination of fermentation, dissolution, and purification steps; and (ii) analyzed by HPLC for the presence of directing molecules, precursor molecules, intermediate molecules, and target molecules such as CBGA, CBGVA, THCA, and THCVA.

In specific embodiments, (a) the host cell produces a meroterpenoid; (b) a second reaction pathway converts the first cannabinoid to a second cannabinoid, as catalyzed by downstream enzymes, which in some embodiments are cannabinoid synthases; (c) the second cannabinoid is THCA, THCVA, CBDA, CBDVA, CBCA, CBCVA, CBGVA or CBGOA; (d) the prenyl donor is polyprenol diphosphate; (e) the prenyl acceptor is a 1,3-diphenol or diphenolic acid; (f) two or more fungal GOT PTs, or a mixture of fungal PTs and plant PTs are present in the host cell; enzymes which catalyze synthesis of the first cannabinoid.

### Methods of Producing Cannabinoids

The present invention is also directed to methods of catalyzing the condensation of a polyprenol diphosphate and an alkylresorcinol or alkylresorcyclic acid to yield a cannabinoid. The methods comprises combining the polyprenol diphosphate and the alkylresorcinol or alkylresorcyclic acid with any of the GOT PTs described above.

In some of these embodiments, the polyprenol diphosphate is geranyl pyrophosphate (GPP) or farnesyl pyrophosphate (FPP) or geranylgeranyl pyrophosphate (GGPP). In other embodiments, the alkylresorcinol or alkylresorcyclic acid is olivetol (OL) or olivetolic acid (OA), divarinolic acid, divarinol, orcinol, orsellinic acid, sphaerolic acid, sphaerophorol. In additional embodiments, the cannabinoid is cannabigerolic acid (CBGA), cannabigerol (CBG), or cannabigerovarinic acid (CBGVA).

The catalysis of these methods may be performed in vitro or in a recombinant microorganism or plant expressing the PT, as described above. In some of these embodiments, the microorganism is a yeast cell, e.g., a species of Saccharomyces, Candida, Pichia, Schizosaccharomyces, Scheffersomyces, Blakeslea, Rhodotorula, or Yarrowia.

In additional embodiments, the recombinant microorganism or plant also expresses a second PT, e.g., a fungal PT and a plant PT, as discussed above.

Also as discussed above, in some embodiments the recombinant microorganism or plant also expresses at least one other recombinant enzyme in a cannabinoid biosynthetic pathway. Nonlimiting examples of the at least one other recombinant enzyme is an olivetolic acid cyclase (OAC), a polyketide synthase, an olivetol synthase, a geranyl pyrophosphate synthase (GPPS), a malonate transporter, an aromatase, a dehydrogenase, an oxidase, a desaturase, a decarboxylase, a cannabinoid synthase including a THCA, a CBCA or a CBDA synthase, a cytochrome P450 (CYP-450), a cytochrome P450 reductase (CPR), or any combination thereof.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1 - Construction of Recombinant Saccharomyces Cerevisiae Strain Expressing a Fungal Prenyltransferase

Construction of Saccharomyces cerevisiae strains expressing a PT with GOT activity derived from a fungal PT for meroterpenoid compound production, such as CBGA, is carried out by cloning the fungal PT into vectors with the proper regulatory elements for gene expression (e.g. promoter, terminator). As an alternative to expression from an episomal plasmid, the PT gene is inserted into the recombinant host genome. Integration is achieved by a single cross-over insertion event of the plasmid. Strains with the integrated gene can be screened by rescue of auxotrophy and genome sequencing.

### Example 2 - Expression of a Mixed Prenyltransferase Pathway for Cannabinoid Production in a Modified Host Organism

A recombinant Saccharomyces cerevisiae host is modified to express multiple PT genes, as illustrated in FIG. 4. A mixture of different PT genes derived from fungi can be expressed, or a mixture of plant-derived PT genes can be expressed along with fungi-derived PT genes in the modified host. Non-limiting examples of plant derived PT genes include those corresponding to GenBank Accession Identification Numbers MO431418.1, PK28436, PK02092, PK17697, PK15523, PK11068, PK13891, PK29226, and PK07278. The optimized genes can be cloned into vectors with the proper regulatory elements for gene expression (e.g. promoter and terminator) and the derived plasmid can be confirmed by DNA sequencing. As an alternative to expression from an episomal plasmid, the optimized PT genes are inserted into the recombinant host genome. Integration is achieved by a single cross-over insertion event of the plasmids. Strains with the integrated genes can be screened by rescue of auxotrophy and genome sequencing.

### Example 3 - Expression of Fungal Cannabinoid Pathway in Conjunction With Downstream Cannabinoid Synthases

Construction of a modified Saccharomyces cerevisiae host is carried out by co-expressing downstream cannabinoid synthases with (i) a fungal-derived PT enzyme, (ii) a mixture of fungal-derived PT enzymes, or (iii) a mixture of fungal derived PT enzymes and plant-derived PT enzymes, as shown in FIG. 4. The recombinant fungal and mixed cannabinoid pathways provide precursors, such as CBGVA, CBGA, and other primary cannabinoids, for downstream cannabinoids synthases to act on. Downstream cannabinoid synthases can include genes encoding for enzymes functioning as THCA synthase, CBDA synthase, CBCA synthase, or other secondary or tertiary cannabinoids. From a primary cannabinoid derived from olivetolic acid (OA) or olivetol (OL), such as CBGA or CBG, downstream cannabinoid synthases may yield THCA, CBDA, CBCA, CBD, CBC, etc. From a primary cannabinoid derived from divarinic acid (DA) or divarinol (DL) such as CBGVA or CBGV, downstream cannabinoid synthases may yield THCVA, THCV, CBDVA, CBDV, CBCVA, CBCV, etc. From a primary cannabinoid derived from orsellinic acid or orcinol, such as CBGOA or CBGO, downstream cannabinoid synthases may yield THCOA, THCO, CBDOA, CBDO, CBCOA, CBCO, etc. From a primary cannabinoid derived from 5-heptylresorcinolic acid (sphaerophorolic acid) or 5-heptylresorcinol (sphaerophorol) such as CBGPA or CBGP, downstream cannabinoid synthases may yield THCPA, THCP, CBDPA, CBDP, CBCPA, CBCP, etc. The optimized downstream cannabinoid synthase genes are synthesized using DNA synthesis techniques known in the art and expressed in a modified host as referenced, as described in PCT Patent Application PCT/US21/36031. Strains with fungal PT and mixed PT pathways co-expressing downstream cannabinoid synthase genes can be screened by rescue of auxotrophy and genome sequencing.

### Example 4 - Method of Growth

Modified host cells which yield cannabinoids, such as the cannabinoids produced by the fungal and mixed PT enzymes described herein, express engineered (i) a fungal-derived PT, (ii) a mixture of fungal-derived PTs, or (iii) a mixture of fungal-derived PTs and plant derived PTs. More specifically, the cannabinoid-producing strain herein is grown in a feedstock as described in U.S. Pat. Application No. 17/068636. An example feedstock use for a modified host expressing the fungal cannabinoid pathway would be growing the strain in a minimal-complete or rich culture media containing yeast nitrogen base, amino acids, vitamins, ammonium sulfate, and a carbon source, such as glucose or molasses. The feedstock is consumed by the modified host which expresses the fungal cannabinoid pathway to convert the feedstock into (i) biomass, (ii) cannabinoids, and (iii) biomass and cannabinoids. Strains expressing the fungal cannabinoid pathway can be grown on feedstock for 12 to 160 hours at 25-37° C. for isolation of the cannabinoid products.

### Example 5 - Detection of Isolated Product

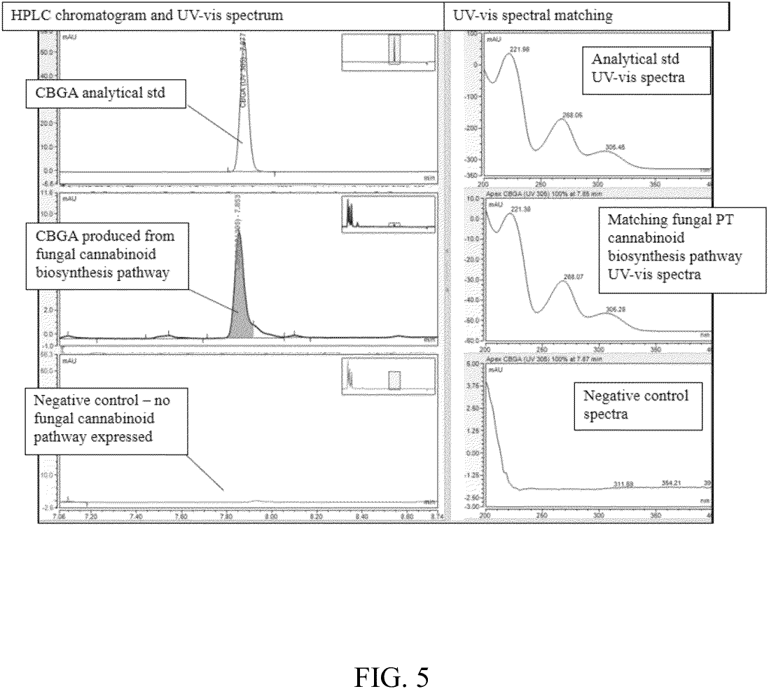

To identify fermentation-derived cannabinoids, such as CBGA, CBGVA, THCVA, THCA, THCV etc. and all other products of a recombinant host expressing an engineered biosynthetic pathway for cannabinoids (see FIGS. 5,6,7 and 8), an Agilent 1100 series liquid chromatography (LC) system equipped with a reverse phase C18 column (Agilent Eclipse Plus C18, Santa Clara, CA, USA) is used. A gradient is used of mobile phase A (ultraviolet (UV) grade H2O + 0.1% formic acid) and mobile phase B (UV grade acetonitrile + 0.1% formic acid). Column temperature is set at 30° C. Compound absorbance is measured at 210 nm and 305 nm using a diode array detector (DAD) and spectral analysis from 200 nm to 400 nm wavelengths. A 0.1 mg/mL analytical standard is made from certified reference material for each cannabinoid (Cayman Chemical Company, USA). Each sample is prepared by diluting fermentation biomass from a recombinant host expressing the engineered biosynthesis pathway 1:3 or 1:20 in 100% acetonitrile and filtered in 0.2 um nanofilter vials. The retention time and UV-visible absorption spectrum (i.e., spectral fingerprint) of the samples are compared to the analytical standard retention time and UV-visible spectra (i.e. spectral fingerprint) when identifying the cannabinoid compounds.

For example, FIG. 5 depicts the detection of CBGA isolated from fermentation with a recombinant host expressing recombinant fungal derived enzymes for CBGA production from OA and GPP. Detection and isolation are depicted by retention time matching of fermentation derived CBGA (middle panel) with a CBGA analytical standard (top panel), along with a matching UV-vis spectral fingerprint of the fermentation derived CBGA with the CBGA analytical standard. This also corroborates that the recombinant host is able to successfully convert OA and GPP to CBGA, which further validates that the systems and methods herein direct molecules into cannabinoid pathways. A negative control (bottom panel), from a strain not expressing the fungal cannabinoid pathway, does not yield CBGA, as seen by no product isolated at the retention time of the CBGA analytical standard, nor a matching UV-vis spectral fingerprint with the analytical standard. This shows selectivity in the isolation and the necessity of expressing the fungal cannabinoid pathway for producing cannabinoid in the modified host strain.

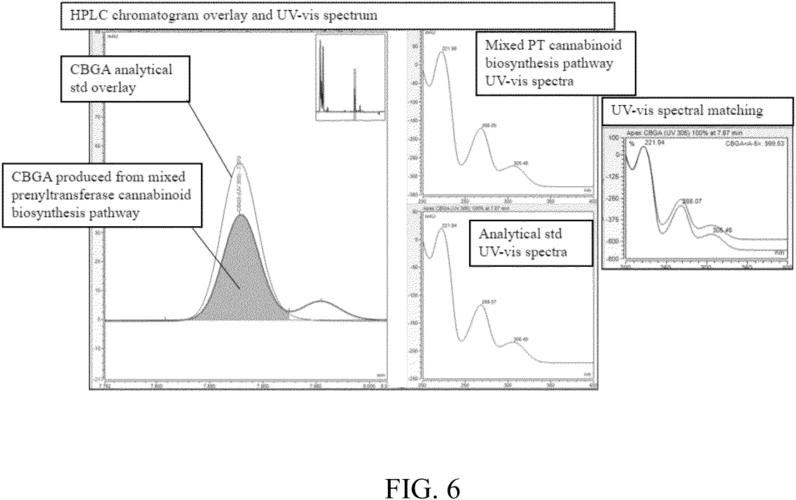

As another example, in FIG. 6, a strain expressing the mixed prenyltransferase cannabinoid pathway diagrammed in FIG. 3 produces CBGA which can be identified and isolated from a fermentation of the modified host. Both retention time and UV-vis absorption spectra (i.e. spectral fingerprint) are identical between the fermentation derived CBGA and the CBGA analytical standard.

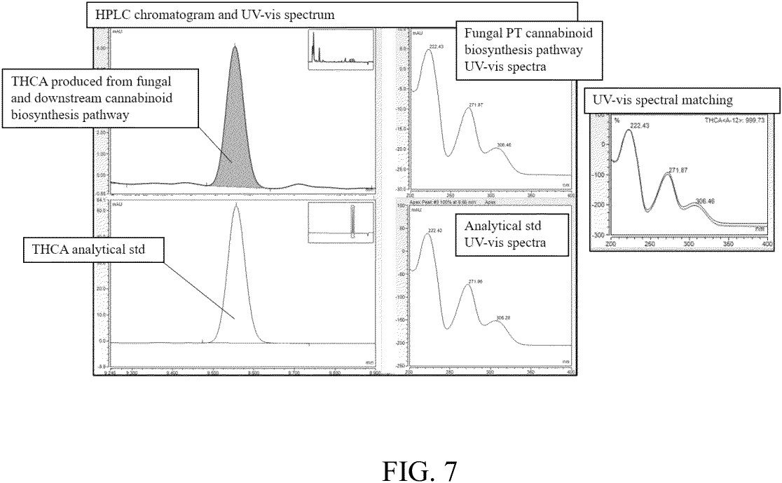

FIG. 7 depicts the production, detection, and isolation of the downstream cannabinoid, THCA, from a fermentation of a modified recombinant host expressing the fungal cannabinoid pathway along with a downstream cannabinoid synthase. The retention time and UV-vis spectral absorption (i.e. spectral fingerprint) of the THCA isolated from fermentation is identical to the retention time and UV-vis spectral absorption (i.e. spectral fingerprint) of the THCA analytical standard. The modified host strain expressing the fungal and downstream cannabinoid pathway is able to produce THCA.

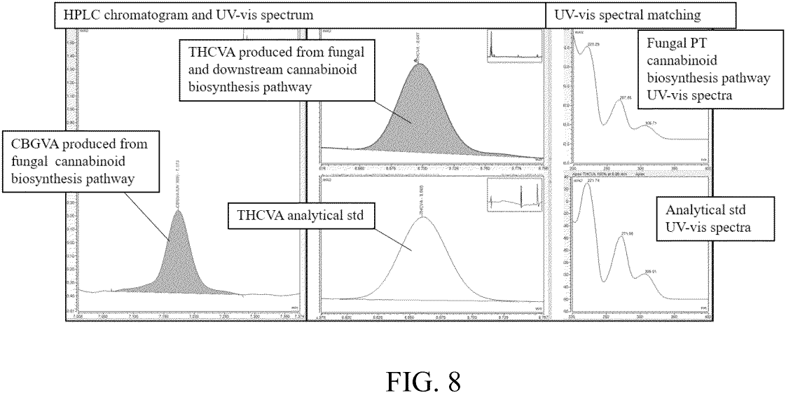

FIG. 8 depicts the production, isolation, and identification of variant cannabinoids, CBGVA, the precursor to THCVA, and THCVA / THCV derived from a fermentation of a recombinant host co-expressing the fungal pathway for cannabinoids and downstream cannabinoid synthases. The primary cannabinoid CBGVA is shown in the left panel. The co-expressed downstream cannabinoid synthase converts CBGVA to a secondary cannabinoid THCVA /THCV, which is identified by matching retention times with the THCVA analytical standard. Spectral library identification of the fermentation derived THCVA matches the UV-vis absorption spectrum of the THCVA analytical standard.

### REFERENCES

Carvalho et al., 2017, FEMS Yeast Res. 17:fox037.
Committee on the Health Effects of Marijuana, 2017, The Health Effects of Cannabis and Cannabinoids: The Current State of Evidence and Recommendations for Research, National Academies Press.
Gülck and Møller, 2020, Trends in Plant Science 25:985-1004.
Green and Sambrook, 2012, Molecular Cloning: A Laboratory Manual (Fourth Edition), Cold Spring Harbor Laboratory Press, Cold Spring Harbor, N.Y.
Luo et al., 2019, Nature 567:123-126.
U.S. Pat. No. 10,435,727.
U.S. Pat. No. 10,671,632.
U.S. Pat. No. 11,041,002.
U.S. Pat. Application 16/553103.
U.S. Pat. Application 16/553120.
U.S. Pat. Application 16/558973.
U.S. Pat. Application 17/068636.
U.S. Provisional Pat. Application 63/035692.
U.S. Provisional Pat. Application 63/053539.
U.S. Provisional Pat. Application 63/141486.
U.S. Provisional Pat. Application 63/164126.
U.S. Provisional Pat. Application 63/194121.
U.S. Pat. Application Publication 2020/0063170.
U.S. Pat. Application Publication 2020/0063171.
http://parts.igem.org/Yeast

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

What is claimed is:

1. A non-naturally occurring nucleic acid comprising a nucleotide sequence encoding a prenyltransferase (PT) protein or a complement of the nucleotide sequence, wherein the nucleotide sequence is codon optimized for production in a microorganism or a plant.

2. (canceled)

3. The nucleic acid of claim 1, wherein the nucleotide sequence is at least 90% identical to any one of SEQ ID NOs: 1-135.

4. The nucleic acid of claim 1, wherein the nucleotide sequence is at least 95% identical to any one SEQ ID NOs: 1-135.

5-6. (canceled)

7. The nucleic acid of claim 1, wherein the nucleotide sequence encodes an enzymatically active PT protein comprising an amino acid sequence having at least 90% amino acid identity to any one of SEQ ID NO: 136-270.

8. The nucleic acid of claim 1, wherein the nucleotide sequence encodes an enzymatically active PT protein comprising an amino acid sequence having at least 95% amino acid identity to any one of SEQ ID NO: 136-270.

9. (canceled)

10. The nucleic acid of claim 3, encoding an enzymatically active PT protein comprising an amino acid sequence having 95% amino acid identity to any one of SEQ ID NO: 136-270.

11. (canceled)

12. The nucleic acid of claim 8, wherein the encoded PT protein comprises one or more conservative amino acid substitutions that prevent or promote a post-translational modification of the PT protein.

13. (canceled)

14. The nucleic acid of claim 1, further comprising another nucleic acid encoding a codon optimized cofolding peptide .

15-16. (canceled)

17. The nucleic acid of claim 14, wherein the codon optimized cofolding peptide is encoded by any one of SEQ ID NOs: 271-275.

18. The nucleic acid of claim 1, wherein the microorganism or plant is a yeast cell, and the nucleic acid further comprises a promoter functional in the yeast cell.

19-20. (canceled)

21. A yeast expression cassette comprising the nucleic acid of claim 1.

22. A non-naturally occurring prenyltransferase (PT) comprising an amino acid sequence having at least 90% amino acid sequence identity to any one of SEQ ID NO: 136-270.

23. The PT of claim 22, comprising the amino acid sequence having at least 95% amino acid sequence identity to any one of SEQ ID NO: 136-270.

24-26. (canceled)

27. The PT of claim 22, expressed in a microorganism or a plant.

28. (canceled)

29. A recombinant microorganism or plant expressing a first PT protein encoded by the nucleic acid of claim 1.

30-31. (canceled)

32. The recombinant microorganism or plant of claim 29, wherein the first PT protein comprises an amino acid sequence having at least 95% amino acid sequence identity or conservative amino acid substitutions to any one of SEQ ID NO: 136-270.

33-34. (canceled)

35. The recombinant microorganism or plant of claim 29, further expressing a second PT protein, the first PT protein is a fungal PT protein and the second PT is a plant PT protein.

36. (canceled)

37. The recombinant microorganism or plant of claim 29, further expressing at least one other recombinant enzyme in a cannabinoid biosynthetic pathway.

38. The recombinant microorganism or plant of claim 37, wherein the at least one other recombinant enzyme is an olivetolic acid cyclase (OAC), a polyketide synthase, an olivetol synthase, a geranyl pyrophosphate synthase (GPPS), a malonate transporter, an aromatase, a dehydrogenase, an oxidase, a desaturase, a decarboxylase, a cannabinoid synthase including a THCA, a CBCA or a CBDA synthase, a cytochrome P450 (CYP-450), a cytochrome P450 reductase (CPR), or any combination thereof.

39. The recombinant microorganism or plant of claim 37, wherein the microorganism or plant:
i) is not a Cannabis sp., and
ii) produces at least one cannabinoid, wherein the at least one cannabinoid is cannabigerol (CBG), cannabigerolic acid (CBGA), cannabidiolic acid (CBDA), cannabichromene (CBC), cannabidivarin (CBCV), cannabichromenic acid (CBCA), cannabichromevarinic acid (CBCVA), cannabinol (CBN), 11-hydroxycannabinol (11-OH-CBN), cannabinerolic acid (CBNA), cannabivarin (CBV), 11-hydroxycannabivarin (11-OH-CBV), cannabinerovarinic acid (CBNVA), cannabigerophorolic acid (CBGPA), cannabigerovarinic acid (CBGVA), cannabigerogerovarinic acid (CBGGVA), tetrahydrocannabinol (THC), tetrahydrocannabinolic acid (THCA), 11-hydroxy tetrahydrocannabinol (11-OH-THC), tetrahydrocannabivarin (THCV), 11-hydroxy tetrahydrocannabivarin (11-OH-THCV), tetrahydrocannabivarin acid (THCVA), cannabinerovarinic acid (CBNVA), sesquicannabigerol (CBF), cannabigerogerol (CBGG), sesquicannabigerolic acid (CBFA), cannabigerogerolic acid (CBGGA), sesquicannabidiolic acid (CBDFA), sesquitetrahydrocannabinolic acid (THCFA), sesquicannabigerovarinic acid (CBFVA), sesquicannabichromenic acid (CBCFA), sesquicannabigerophorolic acid (CBFPA), cannabidiol (CBD), cannabidiolic acid (CBDA), cannabidivarinic acid (CBDVA), cannabidivarin (CBDV) or any combination thereof.

40. (canceled)

41. A method of catalyzing the condensation of a polyprenol diphosphate and an alkylresorcinol or alkylresorcyclic acid to yield a cannabinoid, the method comprising combining the polyprenol diphosphate and the alkylresorcinol or alkylresorcyclic acid with the PT of claim 22 under conditions and for a time sufficient to yield a cannabinoid.

42. The method of claim 41, wherein the polyprenol diphosphate is geranyl pyrophosphate (GPP) or farnesyl pyrophosphate (FPP) or geranylgeranyl pyrophosphate (GGPP), and wherein the alkylresorcinol or alkylresorcyclic acid is olivetol (OL) or olivetolic acid (OA), divarinolic acid, divarinol, orcinol, orsellinic acid, sphaerolic acid, sphaerophorol.

43. (canceled)

44. The method of claim 41, wherein the cannabinoid is cannabigerolic acid (CBGA), cannabigerol (CBG), or cannabigerovarinic acid (CBGVA).

45. The method of claim 41, wherein the catalysis is performed in vitro.

46. The method of claim 41, wherein the catalysis is performed in a recombinant microorganism or plant expressing the PT.

47-48. (canceled)

49. The method of claim 46, wherein the recombinant microorganism or plant also expresses a second PT, and wherein the second PT is a plant PT.

50. (canceled)

51. The method of claim 46, wherein the recombinant microorganism or plant also expresses at least one other recombinant enzyme in a cannabinoid biosynthetic pathway, wherein the at least one other recombinant enzyme is an olivetolic acid cyclase (OAC), a polyketide synthase, an olivetol synthase, a geranyl pyrophosphate synthase (GPPS), a malonate transporter, an aromatase, a dehydrogenase, an oxidase, a desaturase, a decarboxylase, a cannabinoid synthase including a THCA, a CBCA or a CBDA synthase, a cytochrome P450 (CYP-450), a cytochrome P450 reductase (CPR), or any combination thereof.

52. (canceled)

53. The method of claim 51, wherein the microorganism or plant:
i) is not a Cannabis sp., and
ii) produces at least one cannabinoid wherein at least one cannabinoid is cannabigerol (CBG), cannabigerolic acid (CBGA), cannabidiolic acid (CBDA), cannabichromene (CBC), cannabidivarin (CBCV), cannabichromenic acid (CBCA), cannabichromevarinic acid (CBCVA), cannabinol (CBN), 11-hydroxycannabinol (11-OH-CBN), cannabinerolic acid (CBNA), cannabivarin (CBV), 11-hydroxycannabivarin (11-OH-CBV), cannabinerovarinic acid (CBNVA), cannabigerophorolic acid (CBGPA), cannabigerovarinic acid (CBGVA), cannabigerogerovarinic acid (CBGGVA), tetrahydrocannabinol (THC), tetrahydrocannabinolic acid (THCA), 11-hydroxy tetrahydrocannabinol (11-OH-THC), tetrahydrocannabivarin (THCV), 11-hydroxy tetrahydrocannabivarin (11-OH-THCV), tetrahydrocannabivarin acid (THCVA), cannabinerovarinic acid (CBNVA), sesquicannabigerol (CBF), cannabigerogerol (CBGG), sesquicannabigerolic acid (CBFA), cannabigerogerolic acid (CBGGA), sesquicannabidiolic acid (CBDFA), sesquitetrahydrocannabinolic acid (THCFA), sesquicannabigerovarinic acid (CBFVA), sesquicannabichromenic acid (CBCFA), sesquicannabigerophorolic acid (CBFPA), cannabidiol (CBD), cannabidiolic acid (CBDA), cannabidivarinic acid (CBDVA), cannabidivarin (CBDV) or any combination thereof.

54. (canceled)
